(19) United States
(12) Patent Application Publication
(10) Pub. No.: US 2023/0098876 A1
(43) Pub. Date: Mar. 30, 2023

(54) GENERATION OF UTILIZATION MODELS TO IMPROVE SURGICAL BLOCK UTILIZATION

(71) Applicant: CERNER INNOVATION, INC., Kansas City, KS (US)

(72) Inventors: Sanchalita Chatterjee, Tracy, CA (US); Michelle Padgett, Leawood, KS (US); Maria Fernandez Martin, Overland Park, KS (US); Ann Marie McDonough, Duxbury, MA (US)

(21) Appl. No.: 17/484,533

(22) Filed: Sep. 24, 2021

(51) Int. Cl. G16H 40/20; G16H 40/40; G16H 10/65

(52) U.S. Cl. CPC G16H 40/20; G16H 40/40; G16H 10/65

(57) ABSTRACT

Methods, systems, and computer-storage media are provided for a extracting real-time data to generate utilization models and reports for improving the utilization of a surgical block within a healthcare system. Sets of real-time data are extracted from a first source, a second source, and a third source. The sets of data are processed, validated, and transformed into a first set of utilization data. The first set of utilization data is analyzed and a utilization model is generated based on such analysis. Additionally, a report, which includes the utilization model, is generated for display to a user via a user interface. The use of the report and utilization model allow for implementing changes to scheduling and workflow that result in increasing surgical block utilization rates.

### TECHNICAL FIELD

The present subject matter relates, in general, to extracting real-time data and transforming such data into utilization models which can be utilized in improving utilization rates of resources in healthcare systems.

### BACKGROUND

Healthcare systems rely heavily on complex computer systems and servers to communicate and store critical information. As technology has progressed, the cost of healthcare has increased significantly. The management of healthcare systems has become more complex, and new challenges have emerged in balancing the priorities of providing care and remaining profitable. In order to maintain effective and efficient systems, healthcare management systems need to be able to leverage technology that can provide data and analysis of various systems within a healthcare system (e.g. scheduling, surgical scheduling, billing, etc.). However, healthcare management systems lack the ability to extract real-time data from multiple sources that can be processed and used to generate reports for healthcare administrators to implement immediate changes, when needed, in order to increase the effective utilization of various systems and processes within a major healthcare system.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

Healthcare systems, such as hospitals, must be as efficient as possible in order to remain out of debt and continue to provide care for individuals. However, the complex nature of managing a healthcare system in a manner in which the healthcare system can run efficiently and profitably requires not only competent healthcare providers, but various other systems, such as electronic medical record programs in order to run the day to day functions of the healthcare system. Surgical procedures are one of the largest expenses and also areas of profit for a hospital. The cost to complete a surgical procedure can range significantly. Depending on the time the procedure takes, the supplies used, technology needed, and personnel required to effectively run multiple operating rooms and remain profitable is very challenging. As such, each healthcare system ideally needs to run each operating room as efficiently as possible, without diminishing any of the care or safety protocols in place. That said, in order to remain profitable, it is critical to utilize operating rooms in the most efficient manner. This means that each surgical procedure should be booked for the necessary time only and the turnover time between procedures should be managed effectively in order to clean each operating room and prepare it for the next procedure, but also not waste precious time that could be used to complete another procedure.

Currently, monitoring systems in place can provide data to healthcare administrators to analyze, but lack the capability to transform the data and provide such information in real-time. In fact, currently, healthcare systems utilize third party applications in order to track or extract data related to the utilization of operating rooms. The data is then processed and delivered to the healthcare system to analyze options to improve the utilization of the healthcare system's resources. However, because a third party has to collect this data, process it, and deliver it to a healthcare administer, the information presented is not up to date. Often times, the data presented is delayed and may be well over a month old. By the time a healthcare administrator receives such data, it is outdated and may not display an accurate picture of how the healthcare system's resources were utilized. As such, it is difficult to assess the effectiveness of the current systems in place without real-time data. A dynamic tool that is able to extract real-time data from multiple applications within a healthcare management system and provide an analysis without delay to a user, such as a healthcare administer, for use in implementing processes and surgical schedules to promote efficiency and higher utilization rates could greatly improve the present outcomes. Additionally, a tool that would allow for the processing of data from multiple systems within a healthcare management system (e.g. a scheduling application, a surgical procedure application, etc.) in order provide insights into how each resource is used so that the system can then identify, for example, surgical blocks that can be released or re-allocated sooner than originally scheduled, which would provide significant improvements on utilization, profitability, and delivery of prompt care. This will also allow for the proactive management future allocation of surgical blocks in near real-time. The present disclosure provides a such a solution to these challenges and helps identify gaps in surgical block utilization and pro-actively manage surgical block allocation. It is designed to act as a tool for financial, clinical and operational improvement. Additionally, the systems disclosed herein are able to reduce number of interfaces needed to present data and can easily be ported in future on mobile devices which have limited screen space.

At a high level, the present disclosure discloses systems and methods for extracting data in real-time from one or more applications within a healthcare system, processing such data, and generating visual analytics, including utilization models, that provide insight to determine how to improve the utilization rate of a surgical block or understand and remedy why a particular surgical block is failing to perform. The utilization models and analytics are generated in real-time, which provides an accurate picture of current conditions regarding utilization of surgical blocks within a healthcare system. This allows for identification of inefficiencies, such as turnover in operating room delays, where waste may be occurring within scheduled operating blocks. Additionally, the visual analytics and data generated from processing data from multiple sources within the same healthcare management system may also provide opportunities to improve workflow and set up governance and accountability as a performance improvement measure. As opposed to when a third party application or system would extract data related to utilization of surgical blocks, which resulted in lags in presenting any processed data to a healthcare administer, healthcare systems will be able to save both time and money by utilizing the systems described herein. Further, optimizing surgical block utilization may also lead to decrease of healthcare provider burnout, appropriate compensation for healthcare providers, increased profit for the healthcare system while decreasing costs associated with surgical blocks.

Aspects herein describe computer-storage media, computerized methods, and computing systems that are useful in a computer healthcare system by extracting real-time data from multiple sources within the healthcare management system, processing such data, and then generating utilization models and visual analytics that may be used to improve the utilization of surgical blocks, increase profitability, and improve clinical outcomes. In a first aspect, the system extracts a first set of real-time data from a first source during a first predetermined time period, a second set of real-time data from a second source during the first predetermined time period, and a third set of real-time data from a third source during the first predetermined time period. The system then transforms the first set of real-time data, the second set of real-time data, and the third set of real-time data extracted to generate a first set of utilization data. The first set of utilization data is then analyzed and a utilization model is generated from such analysis. The system further generates a report, that includes the utilization model for display to a user via a user interface, wherein implementation of the utilization model increases surgical block utilization rates.

As well, aspects herein are also directed to a system that comprises one or more processors configured to execute computer-readable instructions for an application and cause the application to perform several tasks. First, a first predetermined time period for extracting data from one or more sources is determined. Then, a first set of real-time data from a first source during the first predetermined time period is extracted. A second set of real-time data from a second source during the first predetermined time period is extracted and a third set of real-time data from a third source during the first predetermined time period is also extracted. The system then processes the first set of real-time data, the second set of real-time data, and the third set of real-time data extracted during the first predetermined time period and also validates the first set of real-time data, the second set of real-time data, and the third set of real-time data extracted. Once validated, the system may store the first set of real-time data, the second set of real-time data, and the third set of real-time data extracted in a database. Additionally, the system transforms the first set of real-time data, the second set of real-time data, and the third set of real-time data extracted, wherein transforming the first set of real-time data, the second set of real-time data, and the third set of real-time data extracted comprises producing a first set of utilization data. The first set of utilization data is analyzed and based on such analysis, the system generates a plurality of utilization models and then generates a report that includes the plurality of utilization models for display to a user via a user interface, wherein selection of one or more of the plurality of utilization models increases efficiency of surgical block utilization.

### BRIEF DESCRIPTION OF THE DRAWINGS

Embodiments are described in detail below with reference to the attached drawings figures, wherein:

FIG. 1 is an example system architecture suitable to implement embodiments of the present invention;

FIG. 2 illustrates an example system workflow illustrating implementation of embodiments of the present invention;

FIG. 3 illustrates an example of a generated report comprising a utilization model;

FIG. 4 illustrates an example of another report comprising a utilization model and illustrating the results of analysis of utilization data;

FIG. 5 illustrates another example of a generated report comprising a utilization model and illustrating results of the analysis of utilization data;

FIG. 6 illustrates an example system infrastructure in accordance with aspects

herein;

FIG. 7. illustrates an example flow diagram descripting an exemplary method of executing embodiments of the present invention; and

FIG. 8 is a block diagram of an example computing environment suitable to implement embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

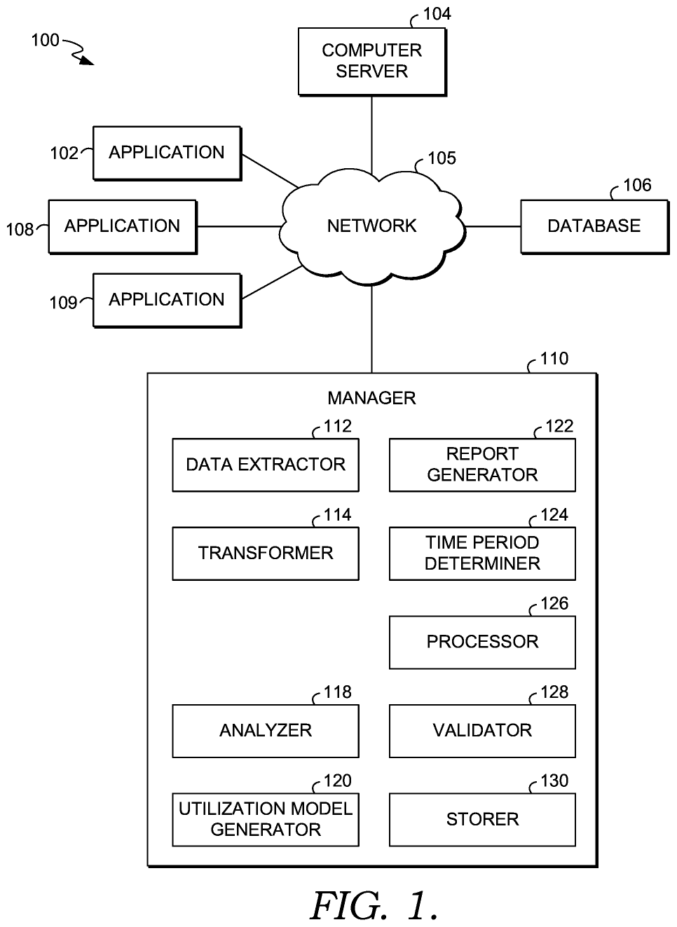

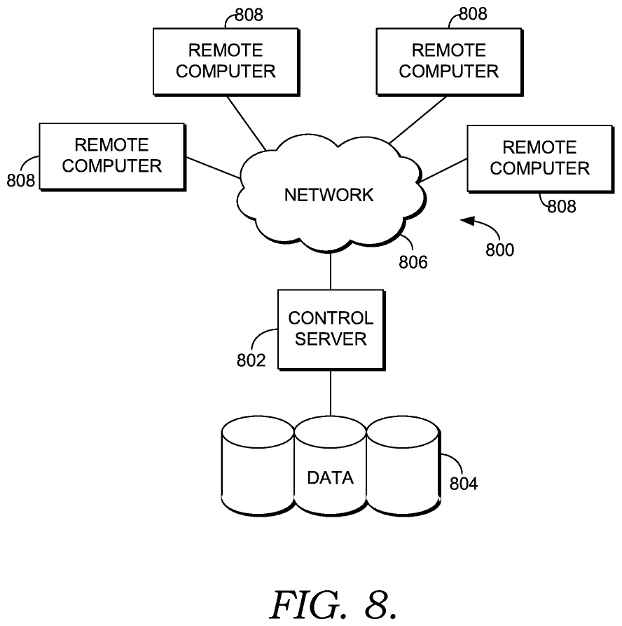

As one skilled in the art will appreciate, embodiments of the present disclosure may be embodied as, among other things: a method, a system, or a set of instructions embodied on one or more computer readable media. Accordingly, the embodiments may take the form of a hardware embodiment, a software embodiment, or an embodiment combining software and hardware. In one embodiment, the invention takes the form of a computer-program product that includes computer-usable instructions embodied on one or more computer readable media, as discussed further with respect to FIGS. 1 and 8.

Embodiments of the present invention are directed to methods, systems, and computer-storage media for utilizing extracted data from multiple sources within the same healthcare management system to generate utilization data, utilization models, and reports that can be utilized to optimize surgical block allocation and use in a healthcare system.

Turning now to FIG. 1, an exemplary computing system 100 is depicted. The computing system 100 (hereinafter “system”) is merely an example of one suitable computing system and is not intended to suggest any limitation as to the scope of use or functionality of embodiments of the present invention. Neither should the system 100 be interpreted as having any dependency or requirement related to any single component or combination of components illustrated herein.

In some embodiments, one or more of the illustrated components may be implemented as a stand-alone application. The components described are exemplary in nature and in number and should not be construed as limiting. Any number of components may be employed to achieve the desired functionality within the scope of the embodiments hereof. Further, components may be located on any number of servers.

In the embodiment shown in FIG. 1, the system 100 includes a manager 110, database 106, network 105, computer server 104, and applications 102, 108, and 109. While FIG. 1 illustrates only one computer server 108, it is contemplated that the system 100 may comprise any number of servers 108.

As shown for exemplary purposes only, the system 100 has three applications, 102, 108, and 109. However, in other aspects, there may be more or less applications present. The applications 102, 108, and 109 may be any type of application that might be used by a user in order to display, review, or analyze data. For example, the application 102 might be an electronic medical record that is comprised of, in exemplary aspects, medication information, vital sign information, physician orders, etc. In some aspects, the applications, 102, 108, and 109 may be specific applications within a particular healthcare management solution system. For example, the application 102 might be a scheduling application that manages patient scheduling and personnel scheduling. The application 108 may be a specialty management application, such as a surgical management application, that manages a variety of data regarding surgical information such as patient information, surgery duration information, surgeon information, and surgery procedure information. In aspects, the application 109 may be another component of a healthcare management solution system, such as a RFID scanner that is used in an operating room to scan computer-readable indicia in order to automatically and electronically log the start time of a procedure, end time of the procedure, room cleansing start time, and/or room cleaning end time. As mentioned, the applications 102, 108, and 109 described are merely examples and it is contemplated that the applications 102, 108, and 109 within system 100 may be any application or source that the system 100 might be in communication with.

Generally, the manager 110 is configured to manage the process of extracting data relevant to improving utilization of surgical block schedules, processing such data to generate utilization data and reports which are transmitted to a user for further determinations related to improving surgical block utilization. As shown, the manager includes a data extractor 112, a transformer 114, an analyzer 118, a utilization model generator 120, a report generator 122, a time period generator 124, a processor 126, a validator 128, and a storer 130. In this aspect, the manager 110 is comprised of nine subcomponents (listed above). However, in other aspects, the manager 110 may be comprised of more or less components and any and all variations are contemplated herein.

Additionally, in some aspects, the manager 110 may also be located within the database 106. It will be appreciated that some or all of the subcomponents of the manager 110 may be accessed via the network 105 and may reside on one or more devices. Further, while system 100 is comprised of one manager 110, it is contemplated that the system 100 may include more than one type of managers and/or multiple instances of the manager 110. It is also contemplated that the manager 110 may be integrated into one or more of the applications 102, 108, and 109.

The data extractor 112 within the manager 110 is configured to extract real-time data from one or more sources, such as applications 102, 108, and 109. The data extractor 112 may extract data from one or more sources based on receiving a signal or instruction or based on a predetermined schedule. For example, upon receiving a signal via network 105, the data extractor 112 extracts specific data based on the signal or instructions received. In aspects, the data extractor 112 will extract multiple sets of real-time data. The data extractor 112 extracts a first set of data from a first source, a second set of data from a second source, and a third set of real-time data from a third source during a predetermined time period. As mentioned, the predetermined time period may be based on a schedule or may be manually instructed.

In system 100, the data extractor 112 extracts a first set of real-time data from a first source, such as the application 102. As mentioned, the application 102 may be a variety of different applications within a healthcare management system. For discussion purposes and as an non-limiting example, the application 102 will be referred to as the first source, which is a surgical management application. The data extractor 112 will also extract a second set of real-time data from a second source, such as the application 108, which for discussion purposes and as a non-limiting example, is a scheduling application. Additionally, the data extractor 112 will extract a third set of data from a third source, such as the application 109, which for discussion purposes and as a non-limiting example, is a RFID scanner utilized in operating rooms. As mentioned, the data extracted by data extractor 112 is real-time data, which means the extraction of such data from each source or application will occur in real-time and without delay. In other words, within a predetermined time period, such as operating room scheduled hours (e.g. 6:00 AM-5:00 PM), the data extractor 112 will extract data for each of the first set of real-time data, second set of real-time data, and third set of real-time data as each source or application is updated to include relevant information. The sets of data may be extracted concurrently or at different times within the predetermined period of time or “window” of time, depending on the source and/or the specific data being extracted.

The predetermined time period is determined by the time period determiner 124. The time period determiner 124 may receive instructions manually via user input or based on historical data, in order to determine characteristics (e.g., a scheduled start time, a scheduled stop time, a predicted duration based on a type of procedure that is scheduled) of the predetermined time period. For example, if a surgical management system, which may assign and manage a specific surgeon's operating schedule and surgical load, changes the type of surgical procedure to be completed and the duration of the particular surgery, the data extractor 112 will immediately extract such information for use. Similarly, if the scheduling system is updated to reflect a change in appointments or general personnel schedule, the data extractor 112 will extract this information in real-time.

Once the data extractor 112 has extracted the first set of real-time data, the second set of real-time data, and the third set of real-time data from corresponding sources, the transformer 114 will transform the first set of real-time data, the second set of real-time data, and the third set of real-time data and generate a first set of utilization data. When the transformer 114 transforms each set of real-time data, various processes occur, such as the validation of the data extracted, utilization of various algorithms that will be used to process the raw data such as procedure time into a useable format for analysis and insight into how to more effectively utilize surgical blocks scheduled. In aspects, the transformation of each set of real-time data by the transformer 114 blends the sets of real-time data and surgery scheduling. In one aspect, the real-time data sets are inserted

Once the transformer 114 has transformed the first set of real-time data, second set of real-time data, and third set of real-time data into the first set of utilization data, the analyzer 118 will analyze the first set of utilization data by utilization of a specific algorithm that is designed to analyze the various data points from each set of real-time data transformed into the first set of utilization data. It is contemplated that the analyzer 118 may analyze the first set of utilization data using an algorithm designed to blend the data from the different sources.

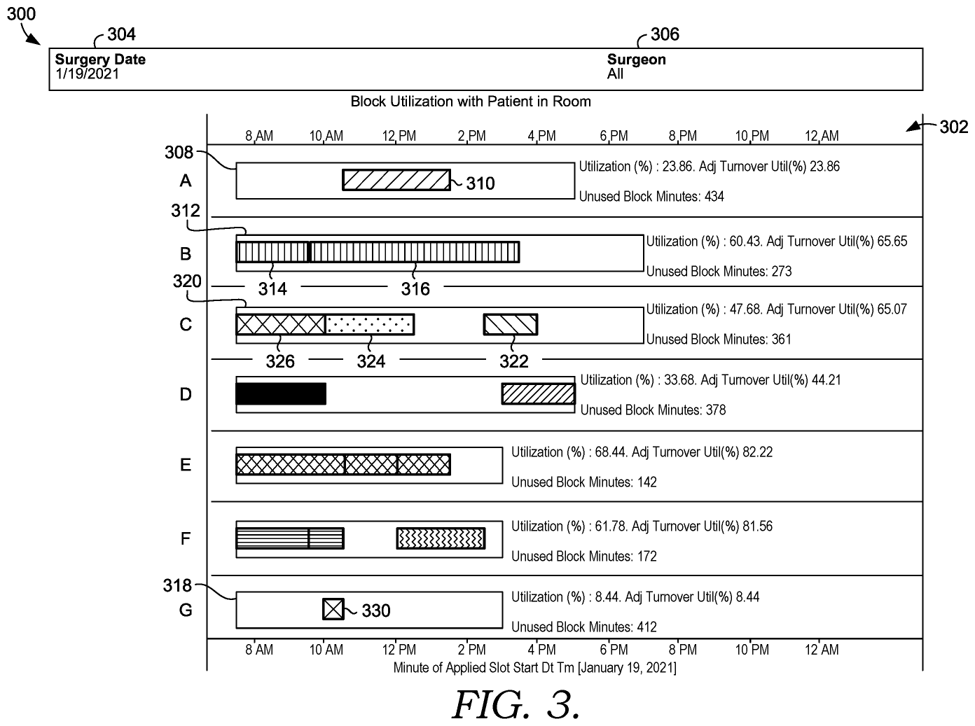

Once the analyzer 118 has analyzed the first set of utilization data, the utilization model generator 120 will generate a utilization model. Such a utilization model will include visual analytics that describe various desired data points and generation of visual analytics that can be used by an individual, such as a healthcare administrator. The utilization model will also include data that was blended by the analyzer 118 and transformed by transformer 114. Examples of utilization models will be further discussed with regard to FIGS. 3-5.

After the utilization model is generated, a report generator 122 will generate a report, which includes the utilization model, for display to a user via a user interface. It is contemplated that the report can include a variety of information, including the utilization rate of each surgical block, turnover time, unused block minutes, used block minutes, and/or visual analytics, such as those shown in FIGS. 3-5. Additionally, the report will include the utilization model generated and any metrics or additional data relevant for making determinations related to surgical block utilization.

The manager 110, in aspects, may also include the processor 126 which can complete various processing steps on each set of data as they are extracted from each source. For example, the processor may be configured to review a first set of data received from a surgical management system and a second set of data received from a scheduling system to eliminate any redundancies and group related data points within each set of data together. The processor 126 may identify multiple data points related to a specific surgeon's scheduled procedure such as surgical procedure information from the surgical application and the specific surgeon's operating schedule for a given day within one or more operating rooms and group this information together so that the analysis of the data by the analyzer 118 is provider specific, if desired.

Additionally, the first set of real-time data, the second set of real-time data, and the third set of real-time data can be validated by the validator 128. To validate, the validator 128 may compare the data points within each set of data and across the different sets of data to other available data within the healthcare management system to confirm the data extracted appears to be both accurate and relevant. For example, the validator 128 may validate the first set of data extracted from the surgical management application by comparing it to previously-extracted similar data that relates to or quantifies the duration of a specific procedure or procedure-specific information. More specifically, the validator 128 can compare the first set of real-time data to a previously-extracted set of real-time data. For example, validator 128 may compare procedure length data from the first set of real-time data for a specific procedure with a particular surgeon to a prior set of data stored that is similar (e.g. same procedure, same surgeon or other overlapping characteristics) to validate the estimated length a procedure may take and determine consistency. The validated data can then be included in the report generated by the report generator 122.

It is also contemplated that throughout the process discussed above, the storer 130 will store data in database 106 for future use. For example, the storer 130 will store the real-time data extracted for each set of data, the utilization data generated, the report generated for the user, including the utilization models generated. This information will be stored in database 106 and can be utilized by the system 100 at any time, as needed. For example, as mentioned, when the validator 128 validates the first set of real-time data based on previous historical data, the previous historical data regarding previous surgical procedures relied upon may be data stored in the database 106 by storer 130.

Once the user receives the report, the user, such as a healthcare administer or surgery director, can update the current surgical block scheduling or settings in order to increase utilization and efficiency by making changes to the current allocation and/or timing of surgical blocks. The updates may be implemented immediately and/or on any scheduled timeframe determined to be appropriate by the user. Additionally, while a first set of data, a second set of data and a third set of data are discussed as being extracted, it is contemplated that the data extractor 112 may extract additional sets of data from each of the first source, second source, and third source for updating the current surgical block schedule. The data extractor 112 may also extract additional sets of data from additional sources if useful in the implementing procedures or updating surgical blocks to improve utilization of surgical blocks. For example, the data extractor could extract additional sets of data related to other medical specialties to coordinate scheduling of necessary testing prior to the scheduled surgical block (e.g. extract a first set of data from an electronic medical record related to laboratory tests and/or radiological imaging records needed prior to the scheduled surgical block and providing this data in the report generated so that the appropriate pre-surgical action is scheduled). Once a surgery schedule is updated in response to the surgical block analysis, the system can further receive an indication of such an update for each operating room that will result in an increase in utilization of that operating room.

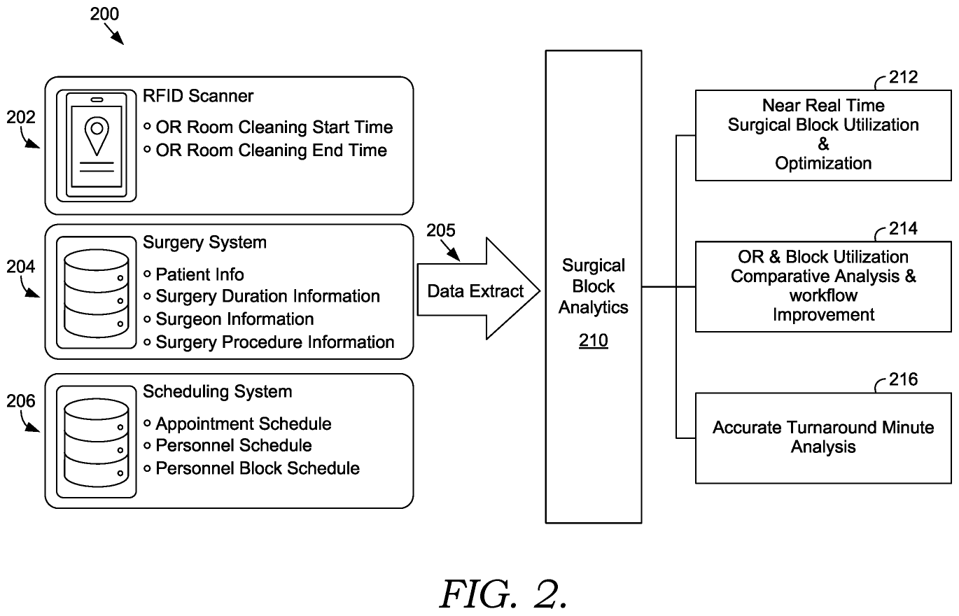

Next, FIG. 2 illustrates an example system workflow 200 illustrating implementation of embodiments of the present invention. As shown, data is extracted at 205 from three sources: RFID Scanner 202, surgery system 204, and scheduling system 206. Upon extraction of the relevant data from each source, surgical block analytics 210, which are performed by the various components of the manager 110 will perform the analytical process discussed above in order to generate the utilization models and reports. As shown in FIG. 2, the reports generated for the user may include near real-time surgical block utilizing and optimization 212, operating room (OR) & block utilization, which may include comparative analyses and workflow improvements 214 and accurate turnaround minute analysis 216. These types of reports are utilized by the user to update workflows and current surgical blocks in order to increase surgical block utilization. While the specific analysis and reports are shown in 212, 214, and 216, it is contemplated that there may be other types of analytical reports generated based on the needs of the healthcare system. The near real-time surgical block utilization and optimization reporting will help eliminate wasted operating room time and manage costs.

Next, FIG. 3 illustrates one example report 300 generated from the by the report generator 122 of FIG. 1 for use in analyzing and implementing improved surgical block utilization.

Report 300 illustrates an analysis of the activity occurring within an example operating room on a particular calendar date 304 (stated as Jan. 19, 2021 as an example). The report 300 includes surgeon information 306, which in this example states “all” meaning that it is encompassing all surgeons utilizing the operating rooms A, B, C, D, E, F, and G on Jan. 21, 2021. In this report 300, the range of time that the operating room could be scheduled is shown at 302 to be from 8:00 AM-12:00 AM (the next day). For example, for the surgery date (Jan. 19, 2021), surgical bocks were scheduled from 8:00 AM on Jan. 19, 2021 through midnight of that same day. The report 300 illustrates multiple surgical blocks for several operating rooms on the surgical date 304. For example, the rectangle or “block” 308 illustrates the scheduled surgical block of time for a procedure from 8:00 AM (scheduled start time) until 5:00 PM (scheduled stop time). The start and stop times of the scheduled surgical time block may be predetermined, preset, and/or may be based on historical usage data. The shaded graphic object, block 310, is displayed in the report 300 as located within the surgical scheduled block 308 in order to represent the time that was used for the particular surgical procedure. The start time, stop time, and duration of block 310 is determined based on the first set of data and second set of data extract from the first and second sources. The report 300 graphically displays the unused minutes or unused amount of time within the scheduled surgical block 308 to illustrate the time unused within the scheduled surgical block 308, which resulted in waste of operating room resources and loss of potential profit. By displaying the procedure duration time blocks inside/within the scheduled surgical blocks, the report 300 provides time waste information at a glance.

While it is critical for surgery blocks that are scheduled to include some buffer or “cushion” time to account for any potential complications occurring during the procedure and/or occurring due to delayed start times, only about a one-third of the scheduled surgical block 308 for operating room A was actually utilized by the procedure shown block 310. This resulted in 434 unused block minutes or about 7 hours of unused time that went to waste, as calculated by the manager 110 and shown in the report 300. The utilization rate for this surgical block for operating room A was calculated by the manager 110 to be 23.86%. By contrast, the scheduled surgical block 312 for operating room B that was scheduled from 8:00 AM until 6:30 PM only had 273 unused block minutes and a higher utilization rate of 60.43%, as determined by the manager 110, relative to operating room A on the same date. Unlike operating room A, operating Room B had two procedures take place back to pack—314 and 316 which also shows more effective utilization of the surgical resources.

Adjusted turnover utilization percentage is based on an algorithm where if the turnover time is greater than 60 minutes, it is adjusted to 60 minutes. For example, if the actual turnover took 86 minutes, the adjusted turnover minutes would be adjusted to 60 minutes. By contrast, if the adjusted turnover minutes were 25 minutes, then the adjusted turnover minutes would remain 25 minutes. An adjusted turnover utilization percentage is then calculated using he adjusted turnover minute determined. in By capturing data through an RFID scanner, such as scanner 202 in FIG. 2, to more accurately capture turnover time since currently the turnover minutes includes idle time between surgery schedules.

Additionally, the graphic pattern and/or color of areas 314 and 316 displayed in the report 300 can be the same in order to visually indicate the same surgeon performed the two procedures. By taking advantage of having the same surgeon perform surgery back-to-back and in the same operating room, the system is able to consolidate resources, save money, and free up another operating room for another surgeon to provide care.

Additionally, the same surgeon performing more than one procedure within the same block may be represented by the same color or patterned block within a scheduled surgical block of time. For example, report 300 has scheduled bock 312 that includes two separate blocks 314 and 316 which illustrate two different surgical procedures conducted by the same surgeon in the same operation room (Room B) on a specific date. Additionally, if a surgeon completes surgery in different operating rooms or in a different scheduled block, the color or pattern assigned to the surgeon may be consistent in each operating room and scheduled block to indicate that the same surgeon was performing surgery at different times in different operating rooms throughout the time captured by the report. It is contemplated that the report 300 generated may be manipulated such that a filter option might be available on the user interface so that a user can filter for a particular surgeon to analyze the surgical block utilization data for a specific surgeon.

A user, such as a surgical director, can utilize the visual analytics and data found in the report 300 in order to determine and implement ways to improve the surgical block utilization, cut costs, and increase profitability for the healthcare system and surgeon. For example, a user could look at the scheduled surgical block 308 and compare it with scheduled surgical block 318 in operating room G and determine whether different workflows and surgical procedures can be scheduled to utilize the resources of both operating rooms A and G more efficiently and effectively. For example, the scheduled surgical block 318 for operating room G had a surgical block scheduled from 8:00 AM until 2:30 PM, but was only used for a shortened duration of time from about 10:00 AM until 10:30 AM, shown by the block 330, resulting in a very poor utilization rate of 8.44%, as determined by the manager 110. Wasting available operating space that could be scheduled for procedures not only costs the healthcare system money, but impacts the care of the population needing treatment as it is likely that operating room G could have been utilized by the same or a different surgeon for at least one more procedure during the scheduled surgical block 318 shown.

A user could utilize the utilization models and data shown in FIG. 3 to update the surgery schedule. For example, operating room A could be scheduled for the next day to accommodate the surgical procedure completed in block 310 and the other surgical procedure completed in block 330 within a shorter duration scheduled block. This would result in an increase in the utilization percentage for operating room A, a decrease in unused block minutes, and open up operating room G to be utilized for at least one more surgical procedure that would bring potential additional profit to the healthcare system, surgeon, and care to someone in need. The value of such utilization models is potentially immeasurable since the data being extracted and analyzed is real-time data presenting an accurate picture of the current state of the surgical block utilization in the healthcare system. The user could use the real-time information and free up an operating room for another procedure that day or at least the next day rather than receiving the utilization data several days to weeks later, when the same factors may no longer be relevant and it is more difficult to accurately adjust for deficiencies. Additionally, being able to dynamically adjust the surgical blocks to increase utilization will save lives and provide surgery to those who otherwise might die waiting for their scheduled surgical procedures.

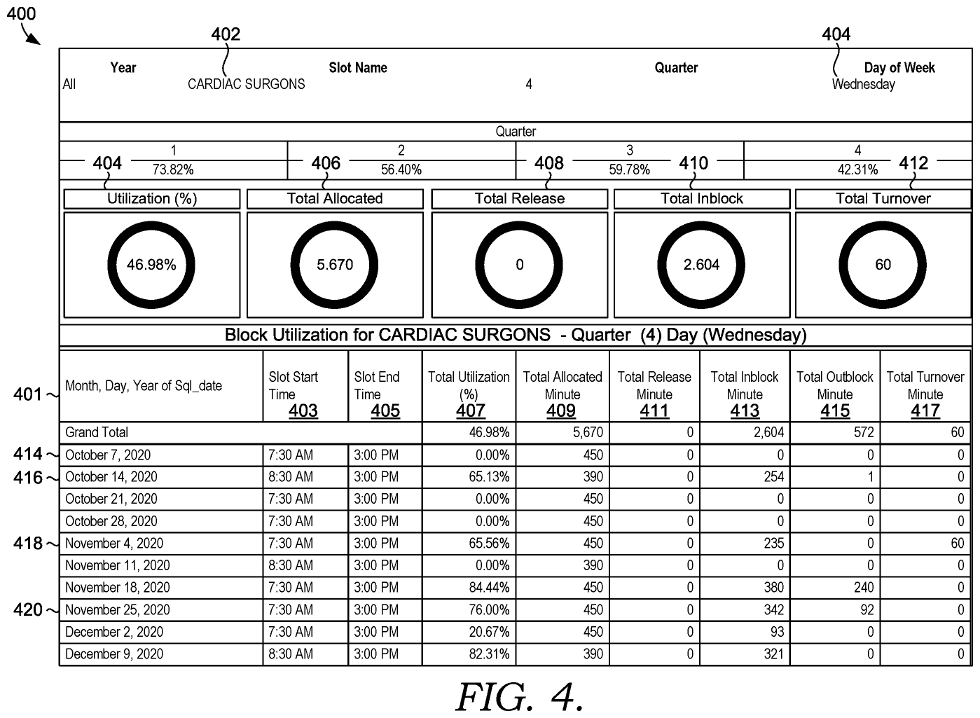

FIG. 4 illustrates another example report 400 generated by the report generator 122. The report 400 utilizes the utilization data and models to generate a block utilization report for cardiac surgeons for one day (Wednesday) of the 4^{th} quarter. As shown, the table includes the month, date, and year of the surgical procedures 401, the slot or surgical block start time 403, the slot end time 405, total allocated minutes 408, total released minutes 411, total in-block minutes (e.g., hours and minutes used for performing the procedure within the scheduled surgical block), total out-of-block minutes (e.g., hours and minutes used for performing the procedure that occur outside of the start time and/or stop time of the scheduled surgical block—for example when a surgery runs past the expected stop time of that scheduled surgical block), and the total turnover minutes 417. As shown for line 414 on Oct. 7, 2020, the surgical block started at 7:30 AM and ended at 3:00 PM. The utilization percentage for this particular date was 0% since none of the total allocated minutes were utilized. What this means is that an operating room remained unused for a whole work day, which resulted in loss of significant money and potentially delayed care for individuals needing surgery. By contrast, line 420 illustrates a date in which the operating surgical block is the same as line 414, but the total allocated minutes 409 were 450 minutes. Of 342 in-block minutes, 43 minutes were utilized and 92 out-of-block minutes 415 occurred, which resulted in a 76.00% utilization rate. This is an improved and more efficient utilization rate than occurred at line 414. The value of this utilization data and report(s) will allow the healthcare administrator to take the real-time data that will be constantly updated and provide insight into ways to increase utilization of the operating rooms.

FIG. 4 further illustrates an average utilization 404 for the time period analyzed (from Oct. 7, 2020-Dec. 9, 2020). Based on the data extracted, the utilization rate for the time period is 46.98%. As such, there is significant room for improvement in order to more efficiently utilize the operating rooms available at the healthcare system and provide better care to individuals in need.

As previously noted, to generate the utilization data, utilization models, and reports illustrated in FIGS. 3-5, the system will extract sets of data from multiple sources, such as a scheduling system, an RFID scanner, and a surgical management system. The real-time data extract is manipulated in various ways and processed or transformed into a set of utilization data which is used to create utilization models like the ones seen in the figures. These models are included in reports generated for a user and displayed on the user interface. The user, such as a surgical director or any other applicable healthcare administrator, is able to analyze the utilization models generated to make almost immediate changes to the surgical block schedule and personnel schedule.

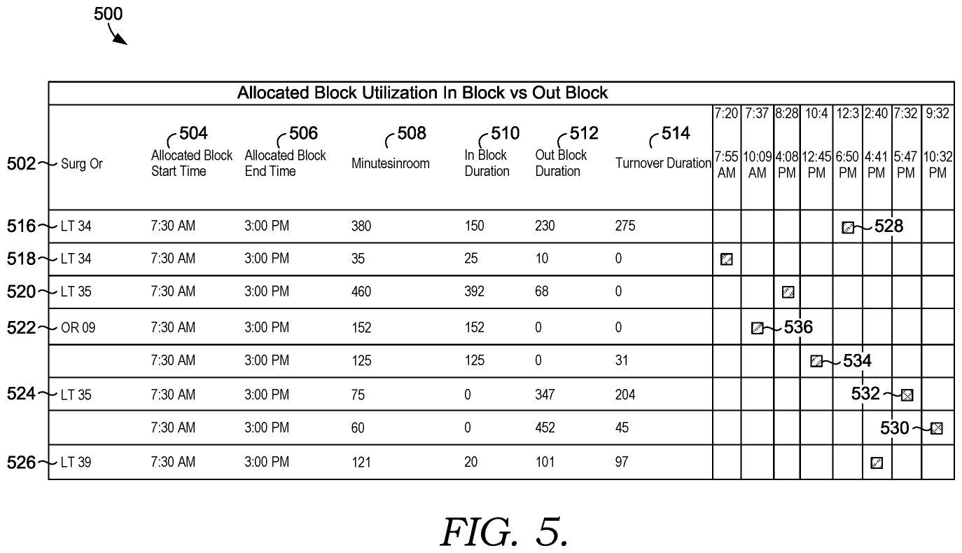

FIG. 5 illustrates another example report including utilization model 500 that may be generated and transmitted to the user for analysis of the utilization rate of the operating rooms. In FIG. 5, the allocated block utilization within the scheduled block is shown in contrast to the utilization out of the scheduled surgical block. Column 502 is configured to show which procedure room is being referenced, such as operating rooms 516, 518, 520, 522, 524, and 526. The allotted block start time 504 and allotted block end time 506 are shown and in this example are consistently from 7:30 AM until 3:00 PM. That said, the allocated block start times and end times may vary day-to-day and may vary per healthcare system. The “minutes in room” 508 is shown for each operating room, including the in-block duration 510 and the out-of-block duration 512. Further, the report includes data about the turnover duration 514 and shows the actual block of time that was utilized. For example, operating room LT 34 was utilized for 380 minutes, of which 150 minutes were in-block (meaning within the scheduled time frame) and 230 minutes were out-of-block (meaning outside the scheduled time frame). There was a turnover duration 514 of 275 minutes. By contrast, operating room 09 was scheduled and used for 152 minutes, resulting in out-of-block time. Patterned block 536 shows the time that the surgical procedure took place and that the time fell within the in-block period. However, when reviewing the utilization data and model for operating room LT 35, there were 347 out-of-block minutes 517 occurring at 5:47 PM. As such, a healthcare administrator could analyze the utilization models to determine ways to increase the utilization rate for the operating rooms based on the allocated in-block and out-of-bock utilization. Once again, the healthcare administrator could then take action and dynamically change the scheduling of the surgical procedures by scheduling procedures more effectively and feeing up previously unused operating room time for other procedures.

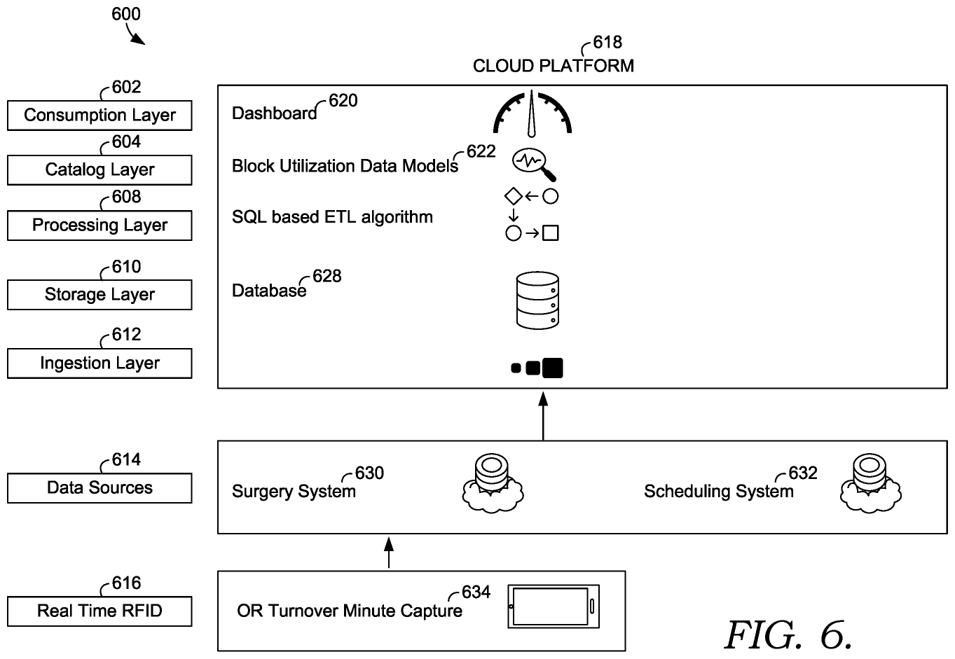

FIG. 6 illustrates an example system infrastructure in accordance with aspects herein. As shown, an RFID scanner 616 provides real-time tracking for a variety of data including the OR start time, end time, and turnover minute capture 634. The ability to extract real-time data from the RFID scanner and utilize that data with data extracted from other sources to process the sets of data and then generate utilization models and reports presents a significant improvement in the field as previously the generation of utilization data required a significant time delay as third party applications that were not a part of the healthcare management system were used to extract the data from the sources on a predetermined schedule such as nightly. By contrast, in the present disclosure, the data from the RFID scanner and any other sources is being transmitted in real-time.

In addition to the data from the RFID 616, additional data sources 614 such as the surgery system 630 and scheduling system 632 are also utilized to extract sets of data applicable to the surgical block analysis. Once the three sets of data have been extracted, they undergo a variety of processing at a first ingestion layer 612. The first ingestion layer 612 allows for the sets of data extracted to be combined or aggregated, validated based on historical data, and for any processing or transforming by application of identified algorithms or other technology to make the sets of data usable for analysis. Once the ingestion or processing is complete, the utilization data is generated, which can be stored in a storage layer 610, such as a database 628. Further processing may be completed at the processing layer 608, in which the utilization data may be further manipulated in order to prepare utilization models. At the catalog layer 604, the utilization data is analyzed and then used to generate block utilization data models such as those models described in FIGS. 3-5. Such models allow for insight into the deficiencies of the current surgical block scheduling system and provide for opportunities to increase utilization, thereby resulting in more effective use of resources, increased profitability, decreased waste, and improved healthcare. Finally, at the consumption layer 602, the reports generated with the utilization models will be illustrated on a user interface or dashboard 620. In some instances, the reports may be generated and sent to a user via email. In other aspects it is contemplated that the reports may be processed and delivered via applications or a healthcare management software or via a user interface that may include a laptop, desktop, tablet, or phone.

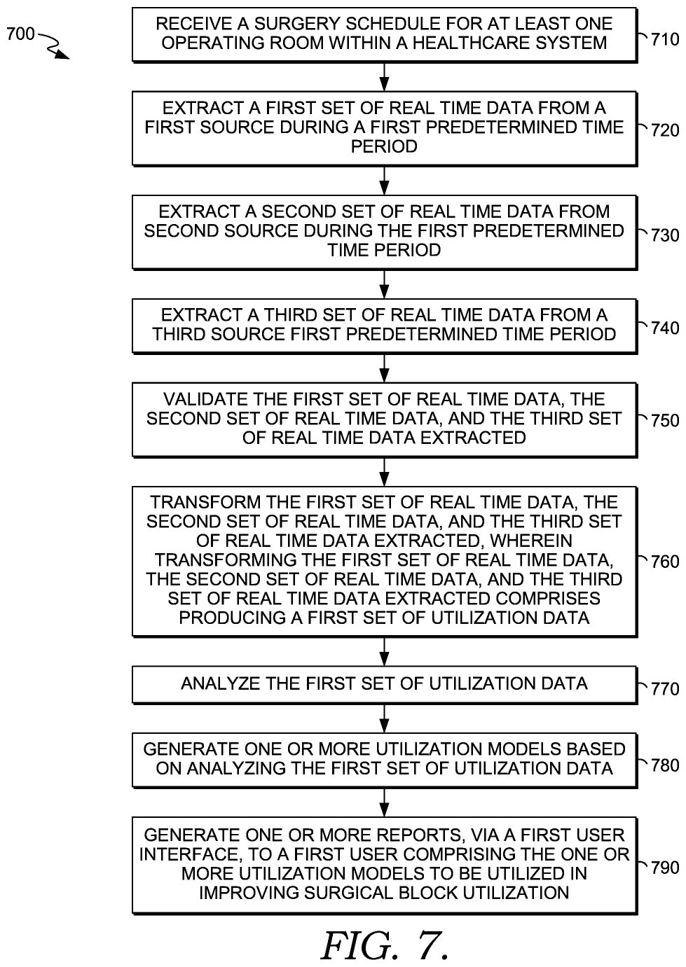

FIG. 7 illustrates an example flow diagram 700 descripting an exemplary method of executing embodiments of the present invention. Beginning with block 710, a surgery schedule is received for at least one operating room within a healthcare system. A data extractor, such as data extractor 112, extracts a first set of real-time data from a first source during a first predetermined time period (e.g., every day, or each day an operating room is scheduled to be used) at block 720. The data extractor also extracts a second set of real-time data from second source during the first predetermined time period at block 730 and then extracts a third set of real-time data from a third source first predetermined time period at block 740. Once the three sets of data have been extracted from the three sources, a validator, such as validator 128, validates the first set of real-time data, the second set of real-time data, and the third set of real-time data extracted at block 750. Then, a transformer transforms the first set of real-time data, the second set of real-time data, and the third set of real-time data extracted, wherein transforming the first set of real-time data, the second set of real-time data, and the third set of real-time data extracted comprises producing a first set of utilization data at block 760. Once the first set of utilization data is produced, the analyzer analyzes the data at block 770. The report generator then generates one or more utilization models based on analyzing the first set of utilization data at block 780. Following this, the report generator generators one or more reports of the one or more utilization models to be displayed to a user via a user interface, wherein implementation of at least one of the utilization models increase efficiency of surgical block utilization at block 790.

An exemplary computing environment suitable for use in implementing embodiments of the present invention is described below. FIG. 8 is an exemplary computing environment (e.g., medical-information computing-system environment) with which embodiments of the present invention may be implemented. The computing environment is illustrated and designated generally as reference numeral 800. The computing environment 800 is merely an example of one suitable computing environment and is not intended to suggest any limitation as to the scope of use or functionality of the invention. Neither should the computing environment 800 be interpreted as having any dependency or requirement relating to any single component or combination of components illustrated therein. It will be appreciated by those having ordinary skill in the art that the connections illustrated in FIG. 8 are also exemplary as other methods, hardware, software, and devices for establishing a communications link between the components, devices, systems, and entities, as shown in FIG. 8, may be utilized in the implementation of the present invention. Although the connections are depicted using one or more solid lines, it will be understood by those having ordinary skill in the art that the exemplary connections of FIG. 8 may be hardwired or wireless, and may use intermediary components that have been omitted or not included in FIG. 8 for simplicity's sake. As such, the absence of components from FIG. 8 should not be interpreted as limiting the present invention to exclude additional components and combination(s) of components. Moreover, though devices and components are represented in FIG. 8 as singular devices and components, it will be appreciated that some embodiments may include a plurality of the devices and components such that FIG. 8 should not be considered as limiting the number of a device or component.

The present technology might be operational with numerous other special-purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 8, the computing environment 800 comprises a computing device in the form of a control server 802. Exemplary components of the control server 802 comprise a processing unit, internal system memory, and a suitable system bus for coupling various system components, including data store 804, with the control server 802. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronics Standards Association (VESA®) local bus, and Peripheral Component Interconnect (PCI) bus, also known as Mezzanine bus.

The control server 802 typically includes therein, or has access to, a variety of non-transitory computer-readable media. Computer-readable media can be any available media that might be accessed by control server 802, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 802. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 802 might operate in a computer network 806 using logical connections to one or more remote computers 808. Remote computers 808 might be located at a variety of locations including operating systems, device drivers and medical information workflows. The remote computers might also be physically located in traditional and nontraditional medical/healthcare care environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other inpatient settings, veterinary environments, ambulatory settings, medical billing and financial offices, hospital administration settings, home healthcare environments, and clinicians' offices. Health care providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 808 might also be physically located in nontraditional medical care environments so that the entire medical community might be capable of integration on the network. The remote computers 808 might be personal computers, servers, routers, network PCs, peer devices, other common network nodes, or the like and might comprise some or all of the elements described above in relation to the control server 802. The devices can be personal digital assistants or other like devices.

Computer networks 806 comprise local area networks (LANs) and/or wide area networks (WANs). Such networking environments are commonplace in offices, enterprise-wide computer networks, intranets, and the Internet. When utilized in a WAN networking environment, the control server 802 might comprise a modem or other means for establishing communications over the WAN, such as the Internet. In a networking environment, program modules or portions thereof might be stored in association with the control server 802, the data store 804, or any of the remote computers 808. For example, various application programs may reside on the memory associated with any one or more of the remote computers 808. It will be appreciated by those of ordinary skill in the art that the network connections shown are exemplary and other means of establishing a communications link between the computers (e.g., control server 802 and remote computers 808) might be utilized.

In operation, an organization might enter commands and information into the control server 802 or convey the commands and information to the control server 802 via one or more of the remote computers 808 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information might also be sent directly from a remote medical device to the control server 802. In addition to a monitor, the control server 802 and/or remote computers 808 might comprise other peripheral output devices, such as speakers and a printer.

Although many other internal components of the control server 802 and the remote computers 808 are not shown, such components and their interconnection are well known. Accordingly, additional details concerning the internal construction of the control server 802 and the remote computers 808 are not further disclosed herein.

The present disclosure has been described in relation to particular aspects, which are intended in all respects to be illustrative rather than restrictive. Further, the present disclosure is not limited to these aspects, but variations and modifications may be made without departing from the scope of the present disclosure.

What is claimed is:

1. A dynamic system that extracts real-time data to generate utilization models and reports for improving the utilization of surgical blocks comprising:
one or more processors configured to:
extract a first set of real-time data from a first source during a first predetermined time period;
extract a second set of real-time data from second source during the first predetermined time period;
extract a third set of real-time data from a third source during the first predetermined time period;
transform the first set of real-time data, the second set of real-time data, and the third set of real-time data extracted to generate a first set of utilization data;
analyze the first set of utilization data;
generate a utilization model based on analyzing the first set of utilization data; and
generating a report, including the utilization model, for display to a user via a user interface, wherein implementation of the utilization model and report results increases surgical block utilization rates.

2. The system of claim 1, wherein the first source is an RFID scanner.

3. The system of claim 1, wherein the first set of real-time data comprises one or more of an room cleaning start time, an room cleaning end time, a procedure start time, or a procedure end time for one or more procedures.

4. The system of claim 1, wherein the second source is a surgical application configured to store data regarding one or more procedures.

5. The system of claim 1, wherein the second set of real-time data comprises one or more of a procedure duration data, a surgery procedure data, individual patient data, or personnel data.

6. The system of claim 1, wherein the third source is a scheduling application.

7. The system of claim 1, wherein the third set of real-time data comprises one or more of an appointment schedule, a personnel schedule, a personnel block schedule, or a room schedule.

8. The system of claim 1, wherein the first set of real-time data, the second set of real-time data, the third set of real-time data, and the first set of utilization data are stored in a database.

9. The system of claim 1, wherein the report generated is an operating room scheduling block utilization report that quantifies a daily utilization of each individual operating room within a healthcare system.

10. The system of claim 9, wherein the daily utilization of each individual operating room includes data indicating a plurality of scheduled surgical blocks, an amount of time that was used for a procedure within each of the plurality of scheduled surgical blocks, a utilization percentage, and a turnover minute analysis.

11. The system of claim 1, wherein the utilization model allows the user to make scheduling updates via the user interface in order to increase efficiency of surgical block utilization.

12. A dynamic system for extracting real-time data to generate utilization models and reports for improving the utilization of surgical blocks comprising:
one or more processors configured to execute computer-readable instructions for an application and to cause the application to:
determine a first predetermined time period for extracting data from one or more sources;
extract a first set of real-time data from a first source during the first predetermined time period;
extract a second set of real-time data from second source during the first predetermined time period;
extract a third set of real-time data from a third source during the first predetermined time period;
process the first set of real-time data, the second set of real-time data, and the third set of real-time data extracted during the first predetermined time period;
validate the first set of real-time data, the second set of real-time data, and the third set of real-time data extracted;
store the first set of real-time data, the second set of real-time data, and the third set of real-time data extracted in a database;
transform the first set of real-time data, the second set of real-time data, and the third set of real-time data extracted, wherein transforming the first set of real-time data, the second set of real-time data, and the third set of real-time data extracted comprises producing a first set of utilization data;
analyze the first set of utilization data;
generate a plurality of utilization models based on analyzing the first set of utilization data; and
generating a report that includes the plurality of utilization models for display to a user via a user interface, wherein selection of one or more of the plurality of utilization models increases surgical block utilization rates.

13. The system of claim 12, wherein the first predetermined time period for extracting data is 4 hours.

14. The system of claim 12, wherein the system extracts an additional set of data from one or more of the first source, the second source, or the third source on a predetermined schedule.

15. The system of claim 12, wherein the system further receives an indication of an updated surgery schedule based on the report generated.

16. The system of claim 15, wherein the updated surgery schedule increases a utilization rate for at least one operating room.

17. The system of claim 12, wherein the report generated comprises data defining “in block” minutes and “out-of-block” minutes for each of a plurality of procedures, wherein “in block” minutes quantify a first duration of time within a scheduled surgical block and wherein “out-of-block” minutes quantify a second duration of time that a procedure extends past the scheduled surgical block.

18. A method comprising:
receiving a surgery schedule for at least one operating room within a healthcare system;
extracting a first set of real-time data from a first source during a first predetermined time period;
extracting a second set of real-time data from second source during the first predetermined time period;
extracting a third set of real-time data from a third source first predetermined time period;
validating the first set of real-time data, the second set of real-time data, and the third set of real-time data extracted;
transforming the first set of real-time data, the second set of real-time data, and the third set of real-time data extracted, wherein transforming the first set of real-time data, the second set of real-time data, and the third set of real-time data extracted comprises producing a first set of utilization data;
analyzing the first set of utilization data;
generating one or more utilization models based on analyzing the first set of utilization data; and
generating one or more reports of the one or more utilization models to be displayed to a user via a user interface, wherein implementation of at least one of the utilization models increase surgical block utilization rates.

19. The method of claim 18, further comprising receiving an updated surgery schedule based on the one or more reports generated to the first user.

20. The method of claim 18, further comprising storing the first set of real-time data, the second set of real-time data, the third set of real-time data, the first set of utilization data, and the one or more reports generated in a database for future use.
